(19) United States
(12) Patent Application Publication
(10) Pub. No.: US 2024/0407866 A1
(43) Pub. Date: Dec. 12, 2024

(54) CONTINUUM ROBOT

(71) Applicant: CANON KABUSHIKI KAISHA, Tokyo (JP)

(72) Inventors: KATSUHIKO OBA, Kanagawa (JP); YUSUKE NIIKAWA, Kanagawa (JP); RYO IWASAWA, Kanagawa (JP); ISAO MATSUOKA, Kanagawa (JP)

(21) Appl. No.: 18/810,080

(22) Filed: Aug. 20, 2024

Related U.S. Application Data
(63) Continuation of application No. PCT/JP2023/003494, filed on Feb. 3, 2023.

(30) Foreign Application Priority Data
Feb. 24, 2022 (JP) 2022-026397

(51) Int. Cl. A61B 34/30; A61B 17/00; A61B 34/20; A61B 90/00; A61M 25/01

(52) U.S. Cl. CPC A61B 34/30; A61B 17/00234; A61B 2017/00292; A61B 2017/00477; A61B 2017/00876; A61B 2034/2059; A61B 2034/301; A61B 2090/064; A61M 25/0113

(57) ABSTRACT

At least one continuum robot is provided, the continuum robot including a linear member having flexibility, a driving unit configured to actuate the linear member, a bendable portion configured to be bent by actuation of the linear member, and a connection device configured to be connected to the linear member and to transmit actuation power of the driving unit to the linear member. The connection device and the linear member are configured to be reconnected after the connection device and the linear member that have been connected are disconnected from each other.

### CROSS-REFERENCE TO RELATED APPLICATIONS

This application is a Continuation of International Patent Application No. PCT/JP2023/003494, filed Feb. 3, 2023, which claims the benefit of Japanese Patent Application No. 2022-026397, filed Feb. 24, 2022, both of which are hereby incorporated by reference herein in their entireties.

### TECHNICAL FIELD

The present disclosure relates to a continuum robot.

### BACKGROUND ART

PTL 1 proposes a curve operation apparatus for endoscope in which a wire connection member is configured to break before a wire breaks. In the curve operation apparatus, when an excessive tension acts on the wire, a breakage-inducing portion of the wire connection member breaks, thereby preventing serious damage, such as breakage of the wire.

### CITATION LIST

### Patent Literature

PTL 1 Japanese Patent Laid-Open No. 2003-339630

However, when the breakage-inducing portion breaks, the power cannot be transmitted, and the power transmission cannot be resumed before repairs are made.

### SUMMARY OF INVENTION

One or more features of the present disclosure provide one or more continuum robots that can resume a power transmission.

According to the present disclosure, a continuum robot includes a linear member having flexibility, a driving unit configured to actuate the linear member, a bendable portion configured to be bent by actuation of the linear member, and a connection device configured to be connected to the linear member and to transmit actuation power of the driving unit to the linear member. The connection device and the linear member are configured to be reconnected after the connection device and the linear member that have been connected are disconnected from each other.

Further features of the present disclosure will become apparent from the following description of exemplary embodiments with reference to the attached drawings.

### BRIEF DESCRIPTION OF DRAWINGS

FIG. 1 is a general view illustrating a medical system.

FIG. 2 is a perspective view illustrating a medical apparatus and a support base.

FIG. 3A is a diagram illustrating a catheter.

FIG. 3B is another diagram illustrating the catheter.

FIG. 4A is a diagram illustrating a catheter unit.

FIG. 4B is another diagram illustrating the catheter unit.

FIG. 5A is a diagram illustrating a base unit and a wire driving unit.

FIG. 5B is another diagram illustrating the base unit and the wire driving unit.

FIG. 5C is another diagram illustrating the base unit and the wire driving unit.

FIG. 6A is a diagram illustrating the wire driving unit and a connection unit.

FIG. 6B is another diagram illustrating the wire driving unit and the connection unit.

FIG. 6C is another diagram illustrating the wire driving unit and the connection unit.

FIG. 7 is a diagram illustrating the wire driving unit.

FIGS. 8A to 8H are diagrams illustrating a connection unit according to a first embodiment.

FIGS. 9A to 9H are diagrams illustrating a connection unit according to a second embodiment.

FIGS. 10A to 10H are diagrams illustrating a connection unit according to a third embodiment.

FIG. 11A is a diagram illustrating a catheter according to a fourth embodiment.

FIG. 11B is another diagram illustrating the catheter unit and the connection unit according to the fourth embodiment.

FIG. 11C is another diagram illustrating the catheter unit and the connection unit according to the fourth embodiment.

FIG. 12A is a diagram illustrating the connection unit according to the fourth embodiment.

FIG. 12B is another diagram illustrating the connection unit according to the fourth embodiment.

FIG. 12C is another diagram illustrating the connection unit according to the fourth embodiment.

### DESCRIPTION OF EMBODIMENTS

Embodiments of the present disclosure will be described with reference to the drawings. Note that dimensions, materials, shapes, and positions, etc., of elements described in the embodiments are to be altered appropriately in accordance with configurations and various conditions of an apparatus to which one or more features of the present disclosure is applied.

### First Embodiment

### <Medical System and Medical Apparatus>

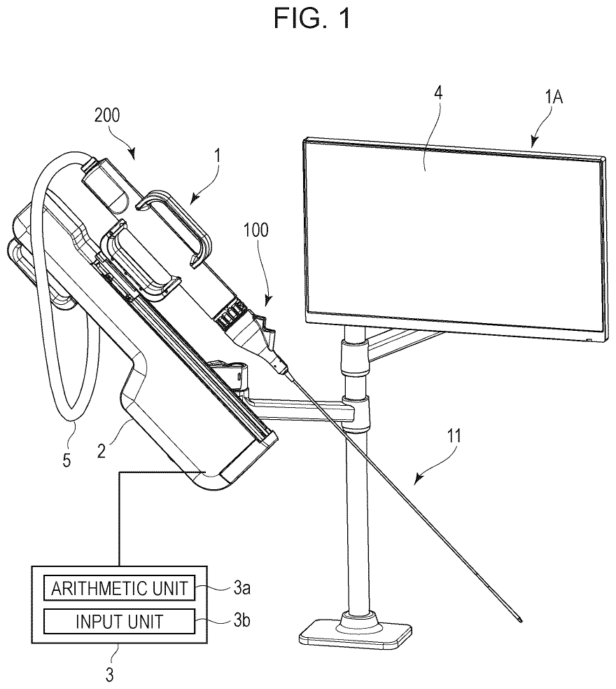

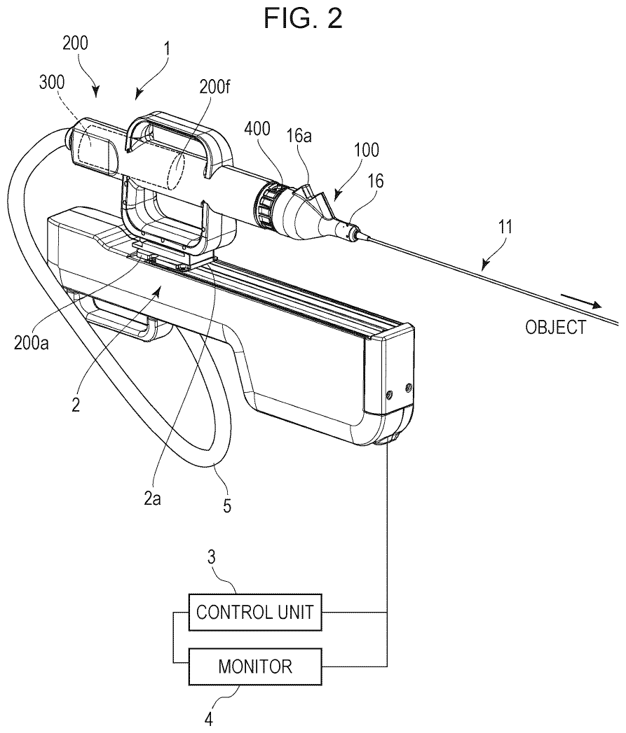

A medical system 1A and a medical apparatus 1 (i.e., continuum robot) will be described with reference to FIGS. 1 and 2. FIG. 1 is a general view illustrating the medical system 1A. FIG. 2 is a perspective view illustrating the medical apparatus 1 and a support base 2.

The medical system 1A includes the medical apparatus 1, the support base 2 on which the medical apparatus 1 is mounted, and a control unit 3 for controlling the medical apparatus 1. In the present embodiment, the medical system 1A also includes a monitor 4 serving as a display unit.

The medical apparatus 1 includes a catheter unit (or bendable unit) 100 and a base unit (or drive unit or installation unit) 200. The catheter unit 100 further includes a catheter 11, which serves as a bendable body. The catheter unit 100 is to be detachably mounted on the base unit 200.

In the present embodiment, a user of the medical system 1A and the medical apparatus 1 inserts the catheter 11 into the inside of an object (or subject or patient) to observe the inside of the object, collect various samples, and give treatment. In some embodiments, the above object is a patient, and the user can insert the catheter 11 into the body of the patient. More specifically, the user inserts the catheter 11 into the bronchus of the patient through the oral or nasal cavity in order to observe lung tissue, collect samples, and remove diseased tissue.

The catheter 11 can be used as a guide device (or a sheath) for guiding medical tools to perform the above work. Examples of the medical tools include an endoscope, forceps, and an abrasion tool. The catheter 11 itself may have functions of such medical tools.

In the present embodiment, the control unit 3 includes an arithmetic unit 3a and an input unit 3b. The input unit 3b receives commands and inputs to operate the catheter 11. The arithmetic unit 3a includes a storage for storing various data and a program for controlling the catheter 11, a random access memory, and a central processing unit for executing the program. The control unit 3 may also include an output unit to output signals for displaying images on the monitor 4.

In the present embodiment, as illustrated in FIG. 2, the medical apparatus 1 is electrically coupled to the control unit 3 via a cable 5 for connecting the base unit 200 of the medical apparatus 1 to the support base 2 and via the support base 2. Note that the medical apparatus 1 can be directly coupled to the control unit 3 by a cable. The medical apparatus 1 can be connected to the control unit 3 via wireless communication.

The base unit 200 of the medical apparatus 1 is detachably mounted on the support base 2. More specifically, the base unit 200 of the medical apparatus 1 has a mounting unit (or attachment unit) 200a, and the mounting unit 200a is detachably mounted on a movable stage (or mounter) 2a of the support base 2. Even when the mounting unit 200a of the medical apparatus 1 is detached from the movable stage 2a, the communication between the medical apparatus 1 and the control unit 3 is maintained so as to enable the control unit 3 to control the medical apparatus 1. In the present embodiment, the medical apparatus 1 and the support base 2 are coupled by the cable 5 even when the mounting unit 200a of the medical apparatus 1 is detached from the movable stage 2a.

When the medical apparatus 1 is detached from the support base 2 (in other words, detached from the movable stage 2a), the user still can move the medical apparatus 1 manually and insert the catheter 11 into the inside of the object.

The user can also use the medical apparatus 1 while the medical apparatus 1 is mounted on the support base 2 and the catheter 11 is inserted in the object. More specifically, while the medical apparatus 1 is mounted on the movable stage 2a, the movable stage 2a moves together with the medical apparatus 1. The medical apparatus 1 can move the catheter 11 in the inserting direction into the object and can withdraw the catheter 11 from the object. The control unit 3 controls the movement of the movable stage 2a.

The mounting unit 200a of the base unit 200 is equipped with a release switch and a detachment switch (not illustrated). When the mounting unit 200a is attached to the movable stage 2a, the user can slide the medical apparatus 1 manually in the guiding direction of the movable stage 2a while the user presses the release switch continuously. The movable stage 2a has a guide for guiding the movement of the medical apparatus 1. When the user stops pressing the release switch, the medical apparatus 1 is fixed to the movable stage 2a. In the state of the mounting unit 200a being mounted on the movable stage 2a, the user can detach the medical apparatus 1 from the movable stage 2a by pressing the detachment switch.

Note that a single switch may have functions of the release switch and the detachment switch. If the release switch is provided with a mechanism that can switch between a pressed state and a non-pressed state, the user does not need to press the release switch continuously when the user slides the medical apparatus 1 manually.

When the mounting unit 200a is attached to the movable stage 2a and neither the release switch nor the detachment switch are pressed, the medical apparatus 1 is fixed to the movable stage 2a, and a motor (not illustrated) moves the movable stage 2a together with the medical apparatus 1.

The medical apparatus 1 includes a wire driving unit (or linear member driving unit, line driving unit, or driving unit) 300 configured to drive the catheter 11. In the present embodiment, the medical apparatus 1 is a robotic catheter apparatus in which the wire driving unit 300 drives the catheter 11 under the control of the control unit 3.

The control unit 3 controls the wire driving unit 300 to bend the catheter 11. In the present embodiment, the wire driving unit 300 is accommodated in the base unit 200. More specifically, the base unit 200 includes a base housing 200f that accommodates the wire driving unit 300. In a word, the base unit 200 includes the wire driving unit 300. The wire driving unit 300 and the base unit 200 are collectively called a “catheter driving apparatus (or base apparatus or main body)”.

In the elongation direction of the catheter 11, the end of the catheter 11 to be inserted into the object is called the “distal end”. In the elongation direction of the catheter 11, the end of the catheter 11 opposite to the distal end is called the “proximal end”.

The catheter unit 100 includes a proximal-end cover 16 that covers the proximal end of the catheter 11. The proximal-end cover 16 has a tool port 16a. A medical tool can be inserted into the catheter 11 through the tool port 16a.

In the present embodiment, as described above, the catheter 11 serves as a guide device that guides a medical tool to a desired position inside the object.

For example, the catheter 11 is inserted to a target position inside the object with an endoscope being accommodated inside the catheter 11. In this case, the user can take either one of actions: to perform manual operation, to move the movable stage 2a, or to drive the catheter 11 using the wire driving unit 300. The endoscope is extracted from the catheter 11 through the tool port 16a after the catheter 11 reaches the target position. Subsequently, another medical tool is inserted through the tool port 16a to collect samples or to give treatment inside the object.

The catheter unit 100 is detachably attached to the catheter driving apparatus (or base apparatus or main body), or more specifically attached to the base unit 200. After the use of the medical apparatus 1, the user can detach the catheter unit 100 from the base unit 200 and attach a new one to the base unit 200 to use the medical apparatus 1 again.

As illustrated in FIG. 2, the medical apparatus 1 includes an operation portion 400. In the present embodiment, the operation portion 400 is included in the catheter unit 100. The user operates the operation portion 400 to attach the catheter unit 100 to the base unit 200 and detach the catheter unit 100 from the base unit 200.

The endoscope inserted in the catheter 11 is coupled to the monitor 4 so that an image captured by the endoscope can be displayed on the monitor 4. The coupling between the control unit 3 and the monitor 4 enables the monitor 4 to display information related to the state or the control of the medical apparatus 1. For example, the monitor 4 can display information related to the position and the navigation of the catheter 11 inside the object. The monitor 4 and the control unit 3 and the endoscope may be coupled by wire or wireless connection. The monitor 4 and control unit 3 may be coupled via the support base 2.

### <Catheter>

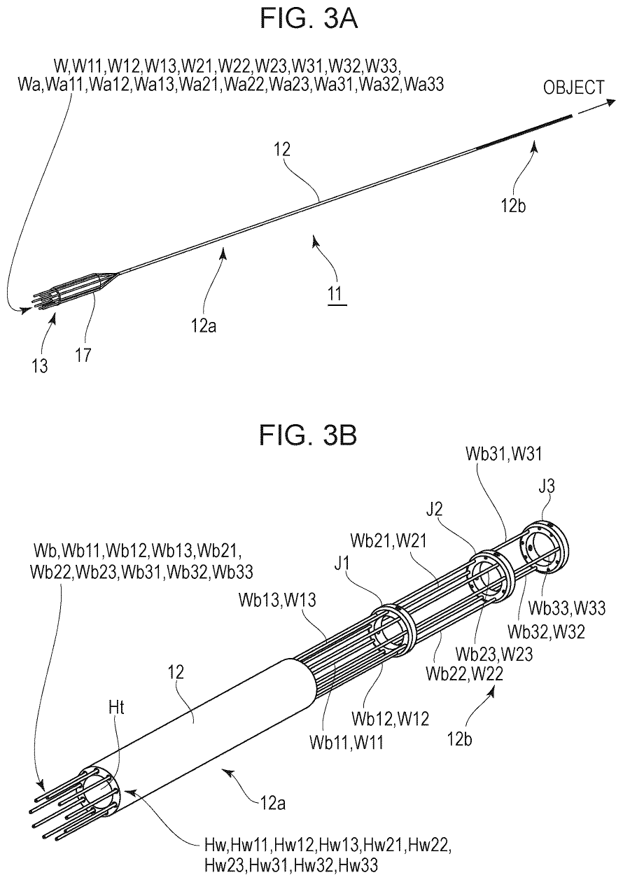

The catheter 11, which serves as the bendable body, will be described with reference to FIGS. 3A and 3B. FIGS. 3A and 3B are diagrams illustrating the catheter 11. FIG. 3A is a general view of the catheter 11. FIG. 3B is an enlarged view of the catheter 11.

The catheter 11 includes a bendable portion (or bendable body or catheter main body) 12 and a bendable-portion driving portion (catheter driving portion) 13 configured to bend the bendable portion 12. The bendable-portion driving portion 13 is configured to receive a driving power from the wire driving unit 300 via a connection unit 21 (to be described later) and thereby bend the bendable portion 12.

The catheter 11 is elongated in the insertion direction of the catheter 11 to be inserted into the object. The elongation direction (or longitudinal direction) of the catheter 11 corresponds to the elongation direction (or longitudinal direction) of the bendable portion 12 and also corresponds to the elongation directions (or longitudinal directions) of first to ninth drive wires W11 to W33, which will be described later.

The bendable-portion driving portion 13 includes multiple drive wires (or drive lines, linear members, or linear actuators) connected to the bendable portion 12. More specifically, the bendable-portion driving portion 13 includes a first drive wire W11, a second drive wire W12, a third drive wire W13, a fourth drive wire W21, a fifth drive wire W22, a sixth drive wire W23, a seventh drive wire W31, an eighth drive wire W32, and a ninth drive wire W33.

Each one of the first to ninth drive wires W11 to W33 includes a flexible wire body (or line body or linear body) Wb. More specifically, the first drive wire W11 includes a first wire body Wb11. The second drive wire W12 includes a second wire body Wb12. The third drive wire W13 includes a third wire body Wb13. The fourth drive wire W21 includes a fourth wire body Wb21.

The fifth drive wire W22 includes a fifth wire body Wb22. The sixth drive wire W23 includes a sixth wire body Wb23. The seventh drive wire W31 includes a seventh wire body Wb31. The eighth drive wire W32 includes an eighth wire body Wb32. The ninth drive wire W33 includes a ninth wire body Wb33.

In the present embodiment, the first to third wire bodies Wb11 to Wb13 are the same in shape. The fourth to sixth wire bodies Wb21 to Wb23 are the same in shape. The seventh to ninth wire bodies Wb31 to Wb33 are the same in shape. In the present embodiment, the first to ninth wire bodies Wb11 to Wb33 are the same in shape except for the length.

First to ninth retention members Wa11 to Wa33 are fixed to respective proximal ends of the first to ninth wire bodies Wb11 to Wb33.

The first to ninth drive wires W11 to W33 are inserted through a wire guide 17 into the bendable portion 12 and are fixed thereto.

In the present embodiment, the first to ninth drive wires W11 to W33 are made of a metal. The material of the first to ninth drive wires W11 to W33 may be a resin. The material of the first to ninth drive wires W11 to W33 may contain a metal and a resin.

An arbitrary one of the first to ninth drive wires W11 to W33 may be referred to as a “drive wire W”. In the present embodiment, the first to ninth drive wires W11 to W33 are the same in shape except for the lengths of the first to ninth wire bodies Wb11 to Wb33.

In the present embodiment, the bendable portion 12 is a flexible tube having a passage Ht through which a medical tool is inserted.

Multiple wire holes are formed in the wall of the bendable portion 12 to enable the first to ninth drive wires W11 to W33 to pass through. More specifically, the wall of the bendable portion 12 has a first wire hole Hw11, a second wire hole Hw12, a third wire hole Hw13, a fourth wire hole Hw21, a fifth wire hole Hw22, a sixth wire hole Hw23, a seventh wire hole Hw31, an eighth wire hole Hw32, and a ninth wire hole Hw33. The first to ninth wire holes Hw11 to Hw33 correspond to the first to ninth drive wire W11 to W33, respectively. The number appears after the reference sign Hw is the same as the number of the corresponding drive wire. For example, the first drive wire W11 is inserted into the first wire hole Hw11.

An arbitrary one of the first to ninth wire holes Hw11 to Hw33 is referred to as a “wire hole Hw”. In the present embodiment, the first to ninth wire holes Hw11 to Hw33 are the same in shape.

The bendable portion 12 has an intermediate section 12a and a bending section 12b. The bending section 12b is positioned in the vicinity of the distal end of the bendable portion 12. A first guide ring J1, a second guide ring J2, and a third guide ring J3 are disposed in the bending section 12b. The bending section 12b is a section in which the bendable-portion driving portion 13 shifts the first guide ring J1, the second guide ring J2, and the third guide ring J3 and thereby controls the direction and the degree of bending of the bendable portion 12. FIG. 3B omits illustration of part of the bendable portion 12 that covers the first to third guide rings J1 to J3.

In the present embodiment, the bendable portion 12 includes multiple auxiliary rings (not illustrated). The first guide ring J1, the second guide ring J2, and the third guide ring J3 are fixed to the wall of the bendable portion 12 in the bending section 12b. In the present embodiment, the auxiliary rings are disposed between the first guide ring J1 and the second guide ring J2 and also between the second guide ring J2 and the third guide ring J3.

The medical tool is guided to the end of the catheter 11 through the passage Ht, the first to third guide rings J1 to J3, and the auxiliary rings.

The first to ninth drive wires W11 to W33 pass the intermediate section 12a and are fixed to corresponding ones of the first to third guide rings J1 to J3.

More specifically, the first drive wire W11, the second drive wire W12, and the third drive wire W13 pass through multiple auxiliary rings and are fixed to the first guide ring J1. The fourth drive wire W21, the fifth drive wire W22, and the sixth drive wire W23 pass through the first guide ring J1 and multiple auxiliary rings and are fixed to the second guide ring J2. The seventh drive wire W31, the eighth drive wire W32, and the ninth drive wire W33 pass through the first guide ring J1, the second guide ring J2, and multiple auxiliary rings and are fixed to the third guide ring J3.

In the medical apparatus 1, the wire driving unit 300 drives the bendable-portion driving portion 13 and thereby bends the bendable portion 12 in directions intersecting the elongation direction of the catheter 11. More specifically, the wire driving unit 300 moves each of the first to ninth drive wires W11 to W33 in the elongation direction of the bendable portion 12 and causes the first to third guide rings J1 to J3 to shift, thereby bending the bending section 12b of the bendable portion 12 in directions intersecting the elongation direction.

The user can insert the catheter 11 to a target position inside the object by taking at least one of the following actions: to move the medical apparatus 1 manually, to move the medical apparatus 1 using the movable stage 2a, and to bend the bendable portion 12.

In the present embodiment, the first to third guide rings J1 to J3 are shifted using the first to ninth drive wires W11 to W33 to bend the bendable portion 12. However, the one or more features and/or embodiments of the present disclosure are not limited to this configuration. One or two of the first to third guide rings J1 to J3 and the corresponding drive wires fixed thereto may be omitted.

For example, the catheter 11 may include the third guide ring J3 only and include the seventh to ninth drive wires W31 to W33 fixed to the third guide ring J3. In other words, the first and second guide rings J1 and J2 and the first to sixth drive wires W11 to W23 may be omitted. Alternatively, the catheter 11 may include the second and third guide rings J2 and J3 only and include the fourth to ninth drive wires W21 to W33 fixed thereto. In other words, the first guide ring J1 and the first to third drive wires W11 to W13 may be omitted.

Alternatively, the catheter 11 may be structured such that one guide ring is driven by two drive wires. In this case, the number of the guide rings may be one or more than one.

### <Catheter Unit>

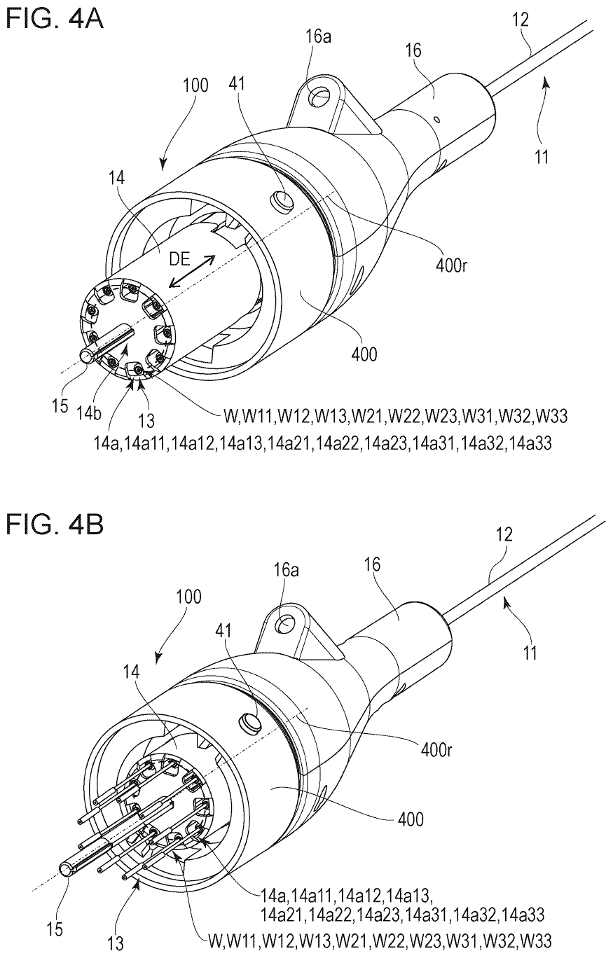

The catheter unit 100 will be described with reference to FIGS. 4A and 4B.

FIGS. 4A and 4B are diagrams illustrating the catheter unit 100. FIG. 4A is a diagram of the catheter unit 100 when a wire cover 14 is at a covering position, which will be described later. FIG. 4B is a diagram of the catheter unit 100 when the wire cover 14 is at a retracted position (or exposing position), which will be described later.

The catheter unit 100 includes the catheter 11 and the proximal-end cover 16. The catheter 11 includes the bendable portion 12 and the bendable-portion driving portion 13, and the proximal-end cover 16 supports the proximal end of the catheter 11. The catheter unit 100 also includes the wire cover 14 that covers and protects multiple drive wires, in other words, the first to ninth drive wires W11 to W33.

The catheter unit 100 is detachably attached to the base unit 200 in the attachment and detachment direction DE. In other words, the attaching direction and the detaching direction of the catheter unit 100 to and from the base unit 200 are parallel to the attachment and detachment direction DE.

The proximal-end cover (or frame, bendable-portion housing, or catheter housing) 16 is a cover that covers part of the catheter 11. The proximal-end cover 16 has the tool port 16a through which a medical tool is inserted into the passage Ht of the bendable portion 12.

Multiple wire-cover holes (or cover holes) are formed in the wire cover 14 to enable the first to ninth drive wires W11 to W33 to pass through. The wire cover 14 has a first wire-cover hole 14a11, a second wire-cover hole 14a12, a third wire-cover hole 14a13, a fourth wire-cover hole 14a21, a fifth wire-cover hole 14a22, a sixth wire-cover hole 14a23, a seventh wire-cover hole 14a31, an eighth wire-cover hole 14a32, and a ninth wire-cover hole 14a33. The first to ninth wire-cover holes 14a11 to 14a33 correspond to the first to ninth drive wire W11 to W33, respectively. The number appears after the reference sign 14a is the same as the number of the corresponding drive wire. For example, the first drive wire W11 is inserted in the first wire-cover hole 14a11.

An arbitrary one of the first to ninth wire-cover holes 14a11 to 14a33 is referred to as a “wire-cover hole 14a”. In the present embodiment, the first to ninth wire-cover holes 14a11 to 14a33 are the same in shape.

The wire cover 14 is movable between a covering position (see FIG. 4A) at which the wire cover 14 covers the first to ninth drive wires W11 to W33 and a retracted position (see FIG. 4B) at which the wire cover 14 is retracted from the covering position. The retracted position is also called an exposing position at which the first to ninth drive wires W11 to W33 are exposed from the wire cover 14.

The wire cover 14 is positioned at the covering position before the catheter unit 100 is attached to the base unit 200. When the catheter unit 100 is attached to the base unit 200, the wire cover 14 moves from the covering position to the retracted position in the attachment and detachment direction DE.

In the present embodiment, the wire cover 14 stays at the retracted position after the wire cover 14 moves from the covering position to the retracted position. Accordingly, once the catheter unit 100 is attached to the base unit 200, the wire cover 14 stays at the retracted position even after the catheter unit 100 is detached from the base unit 200.

The wire cover 14, however, may be structured so as to resume the covering position after the wire cover 14 is moved from the covering position to the retracted position. For example, the catheter unit 100 may have an urging member that urges the wire cover 14 from the retracted position toward the covering position. In this case, when the catheter unit 100 that has been attached to the base unit 200 is detached from the base unit 200, the wire cover 14 is moved from the retracted position to the covering position.

As illustrated in FIG. 4B, the first to ninth drive wires W11 to W33 are arranged along a circle (imaginary circle) having a predetermined radius.

In the present embodiment, the catheter unit 100 includes a key shaft (or key or catheter-side key) 15. In the present embodiment, the key shaft 15 extends in the attachment and detachment direction DE. The wire cover 14 has a shaft hole 14b configured to receive the key shaft 15.

The key shaft 15 is configured to engage a key receptacle 22, which will be described later again. The engagement of the key shaft 15 with the key receptacle 22 restricts, to a predetermined extent, the movement of the catheter unit 100 relative to the base unit 200 in the circumferential direction of the circle (or imaginary circle) along which the first to ninth drive wires W11 to W33 are arranged.

In the present embodiment, as viewed in the attachment and detachment direction DE, the first to ninth drive wires W11 to W33 are arranged so as to surround the key shaft 15. In other words, the key shaft 15 is disposed inside the circle (or imaginary circle) along which the first to ninth drive wires W11 to W33 are arranged. Such arrangement of the key shaft 15 and the first to ninth drive wires W11 to W33 leads to space-saving.

In the present embodiment, the catheter unit 100 includes operation portion 400. The operation portion 400 is movable (rotatable) relative to the proximal-end cover 16 and the bendable-portion driving portion 13.

The operation portion 400 can rotate about a rotation axis (or rotation center) 400r. The rotation axis 400r of the operation portion 400 extends in the attachment and detachment direction DE.

The operation portion 400 is movable (or rotatable) relative to the base unit 200 in the state of the catheter unit 100 being attached to the base unit 200. More specifically, the operation portion 400 is movable (or rotatable) relative to the base housing 200f, the wire driving unit 300, and a connection unit 21 (to be described later).

### <Base Unit>

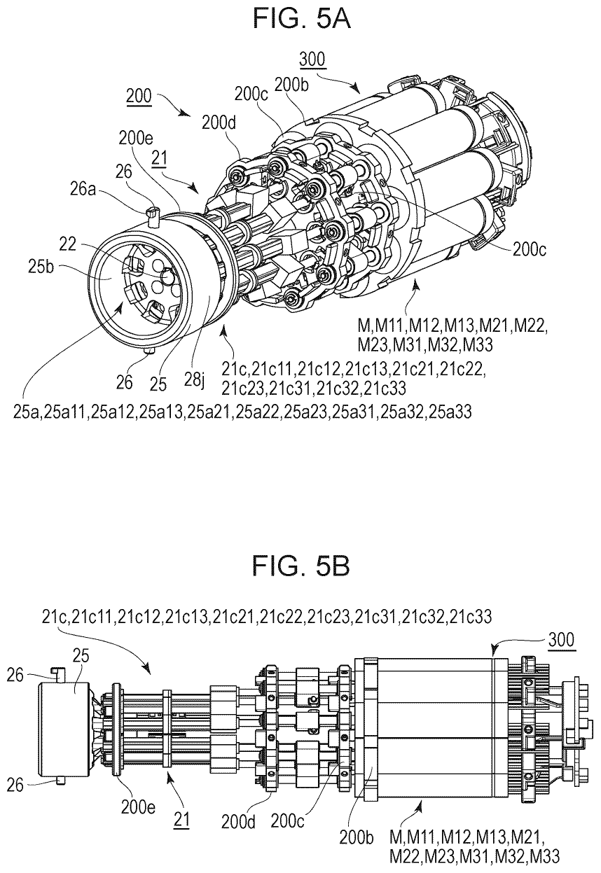

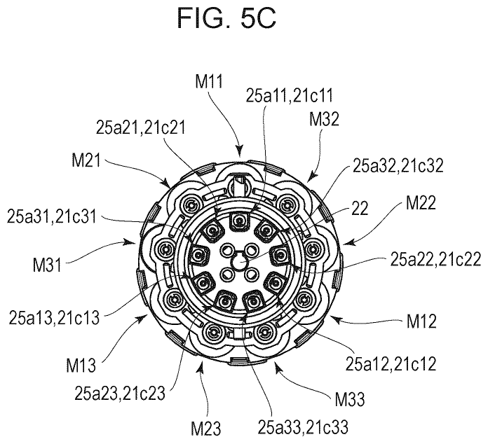

The base unit 200 and the wire driving unit 300 will be described with reference to FIGS. 5A, 5B, and 5C.

FIGS. 5A, 5B, and 5C are diagrams illustrating the base unit 200 and the wire driving unit 300. FIG. 5A is a perspective view illustrating an internal structure of the base unit 200. FIG. 5B is a side view illustrating the internal structure of the base unit 200. FIG. 5C illustrates the base unit 200 as viewed in the attachment and detachment direction DE.

As describe above, the medical apparatus 1 includes the base unit 200 and the wire driving unit 300. In the present embodiment, the wire driving unit 300 is accommodated in the base housing 200f inside the base unit 200. In a word, the base unit 200 includes the wire driving unit 300.

The wire driving unit 300 includes multiple drive sources (motors). In the present embodiment, the wire driving unit 300 includes a first drive source M11, a second drive source M12, a third drive source M13, a fourth drive source M21, a fifth drive source M22, a sixth drive source M23, a seventh drive source M31, an eighth drive source M32, and a ninth drive source M33.

An arbitrary one of the first to ninth drive sources M11 to M33 may be referred to as a “drive source M”. In the present embodiment, the first to ninth drive sources M11 to M33 have the same structure.

The base unit 200 includes the connection unit 21. The connection unit 21 is accommodated in the base housing 200f. The connection unit 21 is connected to the wire driving unit 300. The connection unit 21 includes multiple connection devices. In the present embodiment, the connection unit 21 includes a first connection device 21c11, a second connection device 21c12, a third connection device 21c13, a fourth connection device 21c21, a fifth connection device 21c22, a sixth connection device 21c23, a seventh connection device 21c31, an eighth connection device 21c32, and a ninth connection device 21c33.

An arbitrary one of the first to ninth connection devices 21c11 to 21c33 is referred to as a “connection device 21c”. In the present embodiment, the first to ninth connection devices 21c11 to 21c33 have the same structure.

The connection devices are connected to and driven by respective drive sources. The connection devices transmit the power from the drive sources. More specifically, the first connection device 21c11 is connected to and driven by the first drive source M11. The second connection device 21c12 is connected to and driven by the second drive source M12. The third connection device 21c13 is connected to and driven by the third drive source M13. The fourth connection device 21c21 is connected to and driven by the fourth drive source M21. The fifth connection device 21c22 is connected to and driven by the fifth drive source M22. The sixth connection device 21c23 is connected to and driven by the sixth drive source M23. The seventh connection device 21c31 is connected to and driven by the seventh drive source M31. The eighth connection device 21c32 is connected to and driven by the eighth drive source M32. The ninth connection device 21c33 is connected to and driven by the ninth drive source M33.

The bendable-portion driving portion 13, which includes the first to ninth drive wires W11 to W33, is connected to the connection unit 21, which will be described later. The bendable-portion driving portion 13 receives the driving power from the wire driving unit 300 via the connection unit 21 and thereby bends the bendable portion 12.

The drive wire W has a retention member Wa, and the retention member Wa is connected to the connection device 21c. Multiple drive wires are connected to the respective connection devices.

More specifically, a first retention member Wa11 of the first drive wire W11 is connected to the first connection device 21c11. A second retention member Wa12 of the second drive wire W12 is connected to the second connection device 21c12. A third retention member Wa13 of the third drive wire W13 is connected to the third connection device 21c13. A fourth retention member Wa21 of the fourth drive wire W21 is connected to the fourth connection device 21c21. A fifth retention member Wa22 of the fifth drive wire W22 is connected to the fifth connection device 21c22. A sixth retention member Wa23 of the sixth drive wire W23 is connected to the sixth connection device 21c23. A seventh retention member Wa31 of the seventh drive wire W31 is connected to the seventh connection device 21c31. An eighth retention member Wa32 of the eighth drive wire W32 is connected to the eighth connection device 21c32. A ninth retention member Wa33 of the ninth drive wire W33 is connected to the ninth connection device 21c33.

The base unit 200 includes a base frame 25. Multiple insertion holes are formed in the base frame 25 to enable the first to ninth drive wires W11 to W33 to pass through. The base frame 25 has a first insertion hole 25a11, a second insertion hole 25a12, a third insertion hole 25a13, a fourth insertion hole 25a21, a fifth insertion hole 25a22, a sixth insertion hole 25a23, a seventh insertion hole 25a31, an eighth insertion hole 25a32, and a ninth insertion hole 25a33. The first to ninth insertion holes 25a11 to 25a33 correspond to the first to ninth drive wires W11 to W33, respectively. The number appears after the reference sign 25a is the same as the number of the corresponding drive wire. For example, the first drive wire W11 is inserted into the first insertion hole 25a11.

An arbitrary one of the first to ninth insertion holes 25a11 to 25a33 is referred to as an “insertion hole 25a”. In the present embodiment, the first to ninth insertion holes 25a11 to 25a33 are the same in shape.

The base frame 25 has a mounting cavity 25b into which the wire cover 14 is inserted. The first to ninth insertion holes 25a11 to 25a33 are formed at the bottom of the mounting cavity 25b.

The base unit 200 also includes a main frame 200b, a first bearing frame 200c, a second bearing frame 200d, and a third bearing frame 200e. The main frame 200b, the first bearing frame 200c, the second bearing frame 200d, and the third bearing frame 200e are connected.

The base frame 25 has a key receptacle (or key hole, base-side key, or body-side key) 22 for receiving the key shaft 15. The engagement of the key shaft 15 with the key receptacle 22 enables the catheter unit 100 to join the base unit 200 in correct phase position relative to each other.

The engagement of the key shaft 15 with the key receptacle 22 restricts, to a predetermined extent, the movement of the catheter unit 100 relative to the base unit 200 in the circumferential direction of the circle (or imaginary circle) along which the first to ninth drive wires W11 to W33 are arranged.

As a result, the first to ninth drive wires W11 to W33 pass through the first to ninth insertion holes 25a11 to 25a33 and are connected to the first to ninth connection devices 21c11 to 21c33, respectively. In other words, the drive wire W is prevented from passing through a wrong insertion hole 25 and from being connected to a wrong connection device 21c.

The user can connect the first to ninth drive wires W11 to W33 to respective ones of the first to ninth connection devices 21c11 to 21c33 correctly by inserting the key shaft 15 into the key receptacle 22. Accordingly, the user can attach the catheter unit 100 to the base unit 200 easily.

In the present embodiment, the key shaft 15 has a protrusion protruding in a direction intersecting the attachment and detachment direction DE, and the key receptacle 22 has a recess configured to receive the protrusion. In the circumferential direction, the engagement position between the protrusion and the recess corresponds to the position at which the drive wire W is connected to the correct connection device 21c through the correct insertion hole 25a.

Note that the key shaft 15 can be disposed in ether one of the catheter unit 100 and the base unit 200 and the key receptacle 22 can be disposed in the other. For example, the key shaft 15 may be disposed in the base unit 200, whereas the key receptacle 22 may be disposed in the catheter unit 100.

The base frame 25 includes a locking shaft 26 having a locking nob 26a. The functions of the locking shaft 26 and the locking nob 26a will be described later.

<Connection between Motor and Drive Wire>

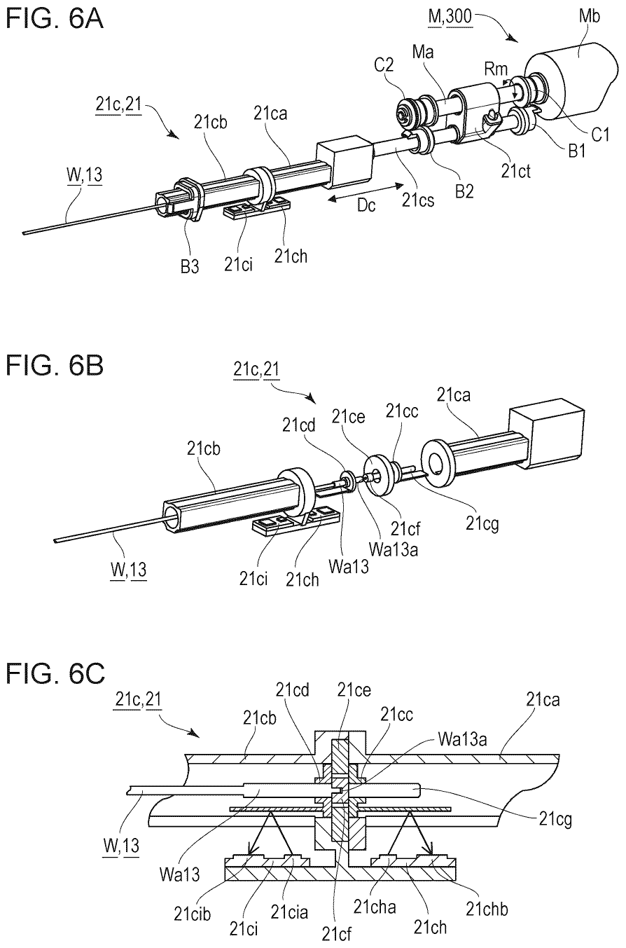

The connection of the wire driving unit 300, the connection unit 21, and the bendable-portion driving portion 13 will be described with reference to FIGS. 6A, 6B, and 6C.

FIGS. 6A, 6B, and 6C are diagrams illustrating the wire driving unit 300 and the connection unit 21. FIG. 6A is a perspective view illustrating the drive source M, the connection device 21c, and the drive wire W. FIG. 6B is an exploded perspective view of the connection device 21c. FIG. 6C is a cross-sectional view of the connection device 21c.

In the present embodiment, the same structure is adopted for respective connections between the first to ninth drive wires W11 to W33 and the first to ninth connection devices 21c11 to 21c33. In addition, the same structure is adopted for respective connections between the first to ninth connection devices 21c11 to 21c33 and the first to ninth drive sources M11 to M33. Accordingly, a typical connection structure will be described using a single drive wire W, a single connection device 21c, and a single drive source M.

As illustrated in FIG. 6A, the drive source M includes a motor shaft Ma and a main body of motor Mb that rotates the motor shaft Ma in a rotation direction Rm. The motor shaft Ma has a helical groove formed at the surface. In other words, the motor shaft Ma is shaped like a screw.

The connection device 21c includes a tractor 21ct connected to the motor shaft Ma and a tractor support shaft 21cs supporting the tractor 21ct. The tractor support shaft 21cs is connected to a first connection base 21ca, and the first connection base 21ca is connected to a second connection base 21cb.

The connection device 21c is supported by a first bearing B1, a second bearing B2, and a third bearing B3. The first bearing B1 is supported by the first bearing frame 200c of the base unit 200. The second bearing B2 is supported by the second bearing frame 200d of the base unit 200. The third bearing B3 is supported by the third bearing frame 200e of the base unit 200. This structure prevents the connection device 21c from rotating about the motor shaft Ma when the motor shaft Ma rotates in the rotation direction Rm. Note that each of the first to ninth connection devices 21c11 to 21c33 has the first bearing B1, the second bearing B2, and the third bearing B3.

When the motor shaft Ma rotates, the helical groove of the motor shaft Ma generates a force exerted on the tractor 21ct in the extending direction of the motor shaft Ma because the connection device 21c is prevented from rotating about the motor shaft Ma. Accordingly, the connection device 21c moves in the extending direction (Dc direction) of the motor shaft Ma. The movement of the connection device 21c causes the drive wire W to move, thereby bending the bendable portion 12.

In other words, the motor shaft Ma and the tractor 21ct constitute a so-called feed screw that converts the rotational motion transmitted from the drive source M to the linear motion. Although the motor shaft Ma and the tractor 21ct constitute a lead-screw actuator in the present embodiment, a ball-screw actuator may be used.

As illustrated in FIGS. 6B and 6C, the first connection base 21ca and the second connection base 21cb accommodate a first connection plate 21cc and a second connection plate 21cd, which serve as connection members, and also accommodate a first magnet 21ce and a second magnet 21cf. An end portion Wa13a of the retention member Wa13 is a magnet. When the catheter unit 100 is attached to the base unit 200, the end portion Wa13a is connected to the second magnet 21cf by magnetism as illustrated in FIG. 6C. The first connection plate 21cc and the second connection plate 21cd are connected to the first magnet 21ce by magnetism.

The first connection plate 21cc is guided by the first connection base 21ca and a guide shaft 21cg, and the second connection plate 21cd is guided by the second connection base 21cb and the retention member Wa13. The guide shaft 21cg is fixed to the second magnet 21cf. The first magnet 21ce is sandwiched and thereby fixed by the first connection base 21ca and the second connection base 21cb.

A first sensor 21ch and a second sensor 21ci are fixed to the second connection base 21cb at a position outside the first connection base 21ca and the second connection base 21cb. The first sensor 21ch and the second sensor 21ci are reflection-type sensors configured to detect the amount of movement of the first connection plate 21cc and the second connection plate 21cd. These sensors may be fixed to the first connection base 21ca. The first sensor 21ch has a light emitter 21cha and a light receiver 21chb. As indicated by the arrow in FIG. 6C, the light emitter 21cha emits light and the light receiver 21chb receives reflected light from the first connection plate 21cc to measure the amount of movement of the first connection plate 21cc. The second sensor 21ci has a light emitter 21cia and a light receiver 21cib. The light emitter 21cia emits light and the light receiver 21cib receives reflected light from the second connection plate 21cd to measure the amount of movement of the second connection plate 21cd. The light described above may be LED light or laser light.

Let N1 denote an attraction force acting between the first connection plate 21cc and the first magnet 21ce, N2 denote an attraction force acting between the second connection plate 21cd and the first magnet 21ce, and N3 denote an attraction force acting between the end portion Wa13a of the retention member Wa13 and the second magnet 21cf. In this case, the relation of N1, N2, and N3 satisfies N1=N2 and N1<N3 (i.e., N2<N3). Accordingly, N3 is greater than N1 and N2. The values of N1, N2, and N3 can be adjusted by adopting different magnets or by changing the area of contact with the magnet.

In the present embodiment, N1 is set to be equal to N2 (i.e., N1=N2). However, N1 may be set to be different from N2 (i.e., N1 #N2). In this case, however, both N1<N3 and N2<N3 need to be satisfied to enable the connection device 21c to operate appropriately (which will be described later).

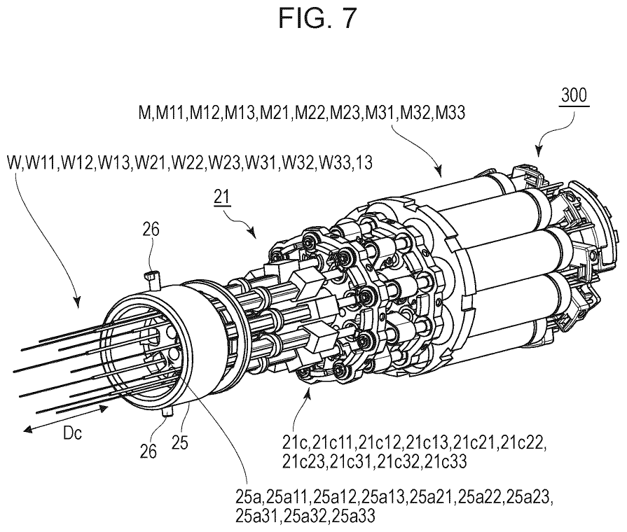

FIG. 7 is a diagram illustrating the connection of the wire driving unit 300, the connection unit 21, and the bendable-portion driving portion 13.

As illustrated in FIG. 7, the first to ninth drive wires W11 to W33 are connected to the first to ninth connection devices 21cii to 21c33, respectively, by attaching the catheter unit 100 to the base unit 200.

The control unit 3 is configured to control each of the first to ninth drive sources M11 to M33 independently. In other words, the control unit 3 can activate or stop any one of the first to ninth drive sources M11 to M33 independently of the other drive sources. Accordingly, the control unit 3 can control each of the first to ninth drive wires W11 to W33 independently. The first to third guide rings J1 to J3 are thereby controlled separately so that the bending section 12b of the bendable portion 12 can be bent in any directions.

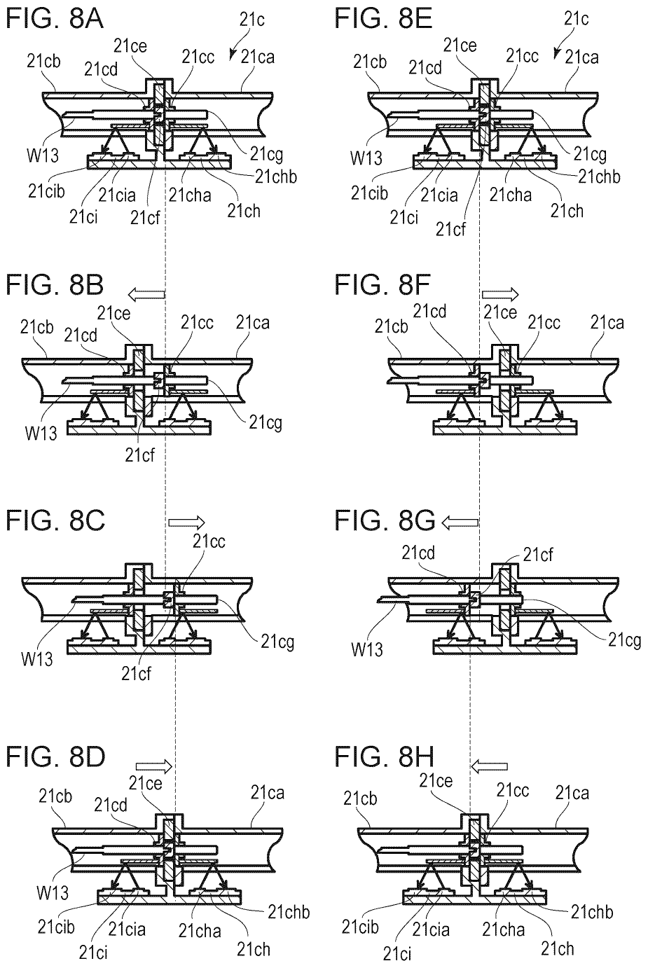

Operation of the connection device 21c of the present embodiment will be described with reference to FIGS. 8A to 8H. FIGS. 8A and 8E illustrate states before the connection device 21c operates. FIGS. 8B, 8C, 8F, and 8G illustrate states after the connection device 21c operates. FIGS. 8D and 8H illustrate states after the connection device 21c returns.

When the catheter 11 is inserted into the inside of the object, the catheter 11 may be caught. In this state, if the user tries to insert the catheter 11 further, an excessive force may act on the caught portion and may cause damage to the object. In this case, it is necessary to remove the excessive force in order to prevent damage to the object. In addition, after the excessive force is removed, it is desirable for the user to resume insertion of the catheter 11 further into the object.

The following describes a procedure of removing the excessive force, returning the catheter 11 to its original state, and resuming the insertion of the catheter 11 further into the object.

FIG. 8A illustrates a state before the connection device 21c starts to operate. The catheter 11 is inserted (leftward in the figure) into the object without generating an excessive force. Here, the distal end of the catheter 11 gets caught inside the object, which generates an excessive force acting, rightward in the figure, on the drive wire W13 and disables the drive wire W13 from proceeding further leftward.

If the user does not aware that the catheter 11 gets caught, the user tries to move the catheter unit 100 and insert the catheter 11 further into the object, which disables the first magnet 21ce from being in contact with the first connection plate 21cc. As a result, the first connection plate 21cc is detached from the first magnet 21ce as illustrated in FIG. 8B. Put another way, if the user tries to insert the catheter 11 further into the object, the first connection base 21ca and the second connection base 21cb proceed leftward in the figure, while the drive wire W13 is prevented from proceeding leftward. As a result, a force is generated so as to separate the first connection plate 21cc from the first magnet 21ce. When the force exceeds N1, the first connection plate 21cc is detached from the first magnet 21ce.

Here, the end portion W13a of the retention member W13 is not separated from the second magnet 21cf since N3 is set to be greater than N1 (i.e., N1<N3) as described above.

Here, any force does not act on the second connection plate 21cd in the direction of the second connection plate 21cd separating from the first magnet 21ce, and accordingly the second connection plate 21cd remains to be in contact with the first magnet 21ce. As illustrated in FIG. 8B, when the first connection plate 21cc is separated from the first magnet 21ce, the first connection base 21ca, the second connection base 21cb, and the components fixed thereto move leftward with respect to the dotted line in the figure.

Simultaneously, the separation of the first connection plate 21cc from the first magnet 21ce removes the excessive force from the drive wire W13. As a result, as illustrated in FIG. 8C, the drive wire W13, the second magnet 21cf, the first connection plate 21cc, and the guide shaft 21cg move rightward with respect to the dotted line in the figure. FIG. 8C illustrates a state in which the excessive force is removed from the drive wire W13 and the tension of the drive wire W13 is relieved.

When the movement occurs as illustrated in FIGS. 8B and 8C, the first sensor 21ch detects the amount of movement of the first connection plate 21cc from the state of FIG. 8A to the state of FIG. 8C. The second sensor 21ci, however, does not detect the movement of the second connection plate 21cd because the second connection plate 21cd does not move relative to the second sensor 21ci. In other words, the second sensor 21ci detects the non-movement of the second connection plate 21cd. The detection of movement of the first connection plate 21cc tells that an excessive force acts on the drive wire W13 and causes the connection device 21c to operate. This information is sent to the control unit 3, and the control unit 3 automatically stops the operation of the wire driving unit 300, thereby preventing damage to the object and displaying a message on the monitor 4 to inform the user that the connection device 21c operates. When the connection device 21c operates while the user inserts the catheter 11 manually, the control unit 3 displays on the monitor 4 that the connection device 21c operates, thereby urging the user to stop manual insertion to prevent damage to the object.

When the connection device 21c operates, the user may use, for example, a button or a lever of the medical system 1A. As a result, as illustrated in FIG. 8D, the control unit 3 activates the wire driving unit 300 to automatically move the first connection base 21ca and the second connection base 21cb and the components fixed thereto rightward in the figure. Since the control unit 3 has obtained the amount of movement of the first connection plate 21cc using the first sensor 21ch, the control unit 3 moves the first connection plate 21cc rightward to the same amount of movement. Accordingly, the control unit 3 stops the wire driving unit 300 when the first connection plate 21cc is attached to the first magnet 21ce and the tension of the drive wire W13 is relieved, which is the same state as that illustrated in FIG. 8A. The connection device 21c returns to the original state. The transmission of power is resumed. The message that the connection device 21c operates disappears from the monitor 4, which enables the user to confirm that the connection device 21c returns to the original state. After the connection device 21c returns to the state in which the tension does not act on the drive wire W13, the user resumes insertion of the catheter 11 into the object so as to avoid an excessive force acting again on the catheter 11 (or the drive wire W13).

The user can insert the catheter 11 into the object so as not to cause damage to the object in the above-described manner. If another excessive force occurs, the user repeats the above procedure to avoid damage to the object and inserts the catheter 11 deeper into the object.

The following describes a procedure of extracting the catheter 11 from the inside of the object.

When the catheter 11 is extracted from the inside of the object, the catheter 11 may be caught. In this state, if the user tries to extract the catheter 11 further, an excessive force may act on the caught portion and may cause damage to the object. In this case, it is necessary to remove the excessive force in order to prevent damage to the object. In addition, after the excessive force is removed, it is desirable for the user to resume extraction of the catheter 11 from the object.

The following describes a procedure of removing the excessive force, returning the catheter 11 to its original state, and resuming the extraction of the catheter 11 from the object.

FIG. 8E illustrates a state before the connection device 21c starts to operate. The catheter 11 is extracted (rightward in the figure) from the object without generating an excessive force. Here, the catheter 11 gets caught inside the object, which generates an excessive force acting, leftward in the figure, on the drive wire W13 and disables the drive wire W13 from proceeding further rightward.

If the user does not aware that the catheter 11 gets caught, the user tries to move the catheter unit 100 and extract the catheter 11 further from the object, which disables the first magnet 21ce from being in contact with the second connection plate 21cd. As a result, the second connection plate 21cd is detached from the first magnet 21ce as illustrated in FIG. 8F. Put another way, if the user tries to extract the catheter 11 further from the object, the first connection base 21ca and the second connection base 21cb proceed rightward in the figure, while the drive wire W13 is prevented from proceeding rightward.

As a result, a force is generated so as to separate the second connection plate 21cd from the first magnet 21ce. When the force exceeds N2, the second connection plate 21cd is detached from the first magnet 21ce.

Here, the end portion W13a of the retention member W13 is not separated from the second magnet 21cf since N3 is set to be greater than N2 (i.e., N2<N3) as described above.

Here, any force does not act on the first connection plate 21cc in the direction of the first connection plate 21cc separating from the first magnet 21ce, and accordingly the first connection plate 21cc remains to be in contact with the first magnet 21ce. As illustrated in FIG. 8F, when the second connection plate 21cd is separated from the first magnet 21ce, the first connection base 21ca, the second connection base 21cb, and the components fixed thereto move rightward with respect to the dotted line in the figure.

Simultaneously, the separation of the second connection plate 21cd from the first magnet 21ce removes the excessive force from the drive wire W13. As a result, as illustrated in FIG. 8G, the drive wire W13, the second magnet 21cf, the second connection plate 21cd, and the guide shaft 21cg move leftward with respect to the dotted line in the figure. FIG. 8G illustrates a state in which the excessive force is removed from the drive wire W13 and the tension of the drive wire W13 is relieved.

When the movement occurs as illustrated in FIGS. 8F and 8G, the second sensor 21ci detects the amount of movement of the second connection plate 21cd from the state of FIG. 8E to the state of FIG. 8G. The first sensor 21ch, however, does not detect the movement of the first connection plate 21cc because the first connection plate 21cc does not move relative to the first sensor 21ch. In other words, the first sensor 21ch detects the non-movement of the first connection plate 21cc. The detection of movement of the second connection plate 21cd tells that an excessive force acts on the drive wire W13 and causes the connection device 21c to operate. This information is sent to the control unit 3, and the control unit 3 automatically stops the operation of the wire driving unit 300, thereby preventing damage to the object and displaying a message on the monitor 4 to inform the user that the connection device 21c operates. When the connection device 21c operates while the user extracts the catheter 11 manually, the control unit 3 displays on the monitor 4 that the connection device 21c operates, thereby urging the user to stop manual extraction to prevent damage to the object.

When the connection device 21c operates, the user may use, for example, a button or a lever of the medical system 1A. As a result, as illustrated in FIG. 8H, the control unit 3 activates the wire driving unit 300 to automatically move the first connection base 21ca and the second connection base 21cb and the components fixed thereto leftward in the figure. Since the control unit 3 has obtained the amount of movement of the second connection plate 21cd using the second sensor 21ci, the control unit 3 moves the second connection plate 21cd leftward to the same amount of movement. Accordingly, the control unit 3 stops the wire driving unit 300 when the second connection plate 21cd is attached to the first magnet 21ce and the tension of the drive wire W13 is relieved, which is the same state as that illustrated in FIG. 8E. The connection device 21c returns to the original state. The transmission of power is resumed. The message that the connection device 21c operates disappears from the monitor 4. After the connection device 21c returns to the state in which the tension does not act on the drive wire W13, the user resumes extraction of the catheter 11 from the object so as to avoid an excessive force acting again on the catheter 11 (or the drive wire W13). After the connection device 21c returns to the original state, it is desirable to manipulate the catheter 11 differently from the way done before the connection device 21c returns to the original state.

Taking the same action as that before the connection device 21c returns may cause the connection device 21c to operate again.

The user extracts the catheter 11 from the object so as not to cause damage to the object in the above-described manner. If another excessive force occurs, the user repeats the above procedure to avoid damage to the object and extracts the catheter 11 further from the object.

The operation of the connection device 21c has been described using the drive wire W13. The first to ninth drive wires W11 to W33 are configured in the same manner.

In the present embodiment, the sensors used in the operation of the connection device 21c are optical sensors. The sensors, however, may be pressure sensors configured to detect the contact between the first magnet 21ce and the first and second connection plates 21cc and 21cd when the connection device 21c returns to the original state.

### Second Embodiment

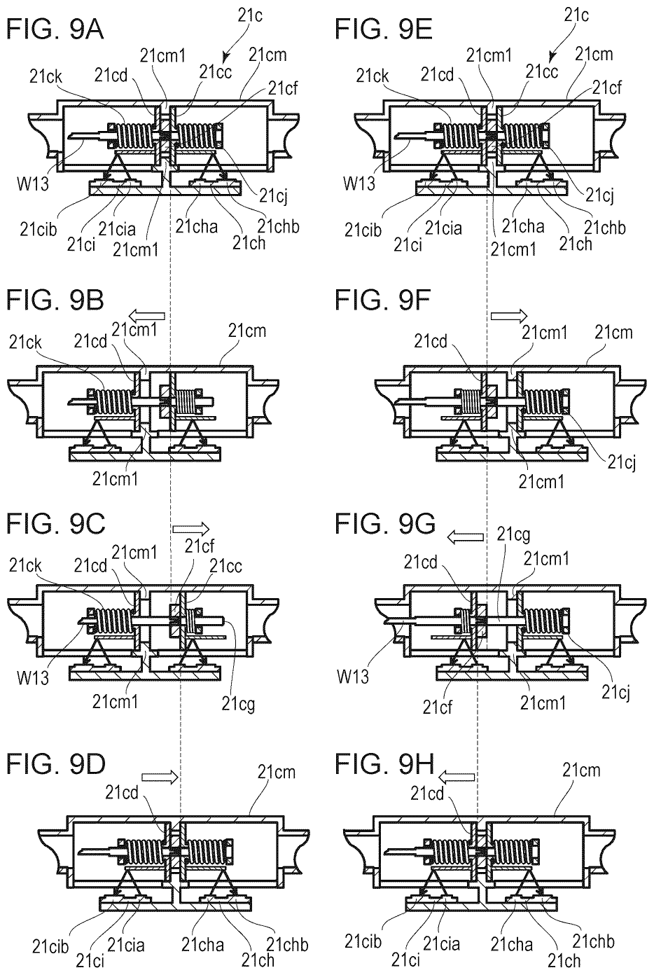

A connection device 21c of the present embodiment will be described with reference to FIGS. 9A to 9H. FIGS. 9A and 9E illustrate states before the connection device 21c operates. FIGS. 9B, 9C, 9F, and 9G illustrate states after the connection device 21c operates. FIGS. 9D and 9H illustrate states after the connection device 21c returns. The states illustrated in FIGS. 9A to 9H correspond to the states illustrated in FIGS. 8A to 8H, which occur in the same timing.

In the present embodiment, the second magnet 21cf is fixed to the drive wire W13.

In the first embodiment, the connection unit 21 also serves to connect the catheter unit 100 to the base unit 200. In the present embodiment, however, the connection between the catheter unit 100 and the base unit 200 is achieved by other members. In other words, the connection device 21c illustrated in FIGS. 9A to 9H does not serve to connect the catheter unit 100 to the base unit 200. The illustration of the connection portion between the catheter unit 100 and the base unit 200 is not provided.

The first magnet 21ce, which is included in the connection device 21c in the first embodiment, is not used for the connection between the first and second connection plates. For example, the second connection plate 21cd is moved leftward in the states illustrated in FIGS. 8B and 8C. If the first magnet 21ce is not provided, the second connection plate 21cd may not return to the state of FIG. 8A when the connection device 21c returns to the state of FIG. 8D. The same applies to the case in which the first connection plate 21cc is moved in the states illustrated in FIGS. 8F and 8G. In order to solve this problem, as illustrated in FIGS. 9A to 9H, the connection device 21c of the present embodiment includes a first pressure spring 21cj and a second pressure spring 21ck, both of which are elastic members. The first pressure spring 21cj and the second pressure spring 21ck urge the drive wire W13 in the direction of the drive wire W13 being connected to the connection device 21c. In the states illustrated in FIGS. 9B and 9C, the second pressure spring 21ck urges the second connection plate 21cd to abut an abutment rib 21cm1 of a connection base 21cm. Accordingly, when the connection device 21c comes to the state illustrated in FIG. 9D, the connection device 21c can return to the original state of FIG. 9A since the second connection plate 21cd does not move. The same applies to the states illustrated in FIGS. 9F and 9G. The first pressure spring 21cj prevents the first connection plate 21cc from moving. Accordingly, when the connection device 21c comes to the state illustrated in FIG. 9H, the connection device 21c can return to the original state of FIG. 9E.

In the present embodiment, spring forces act on the first connection plate 21cc and the second connection plate 21cd, and accordingly the level of magnetism is set appropriately by taking the spring forces into account. The other structures and operations of the connection device 21c are the same as described in the first embodiment, and duplicated descriptions are omitted.

The catheter 11 as configured above is inserted into or extracted from the object without causing damage to the object. If an excessive force occurs again, the user repeats the above procedure to avoid damage to the object and inserts the catheter 11 into the object or extracts the catheter 11 from the object.

Also in the present embodiment, when an excessive force applies to any of the first to ninth drive wires W1l to W33, the user takes the above-described actions to avoid damage to the object and continues to operate the catheter 11.

Although the connection device 21c is disposed in the catheter unit 100 in the present embodiment, the connection device 21c may be disposed in the base unit 200.

In FIGS. 9A to 9H, the dotted line is provided for the purpose of facilitating clear understanding of movement of the components of the connection device 21c.

The first to ninth drive wires W11 to W33 are configured in the same manner also in the present embodiment.

### Third Embodiment

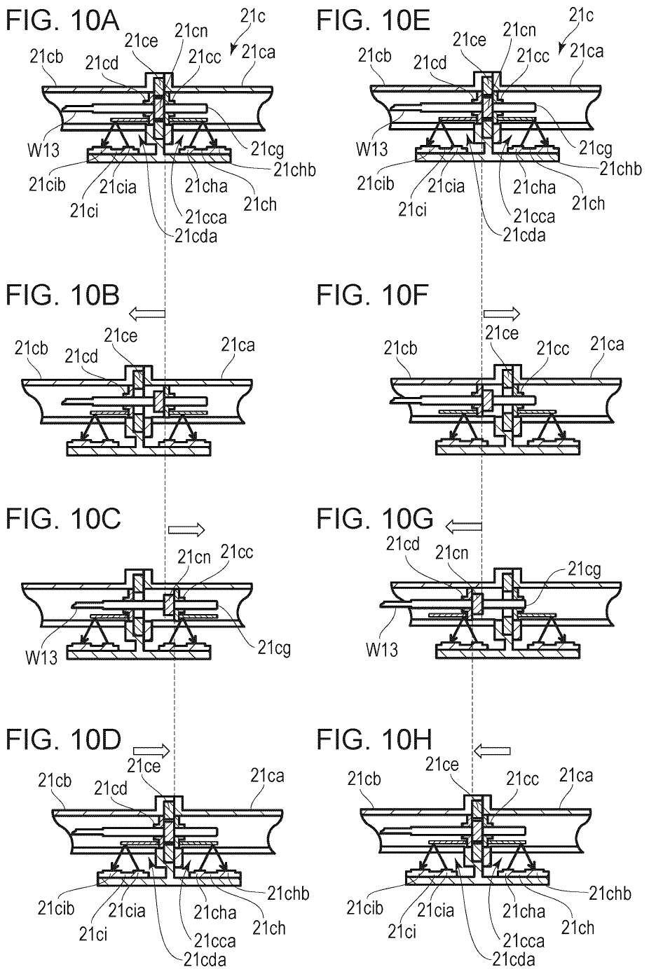

A connection device 21c of the present embodiment will be described with reference to FIGS. 10A to 10H. FIGS. 10A and 10E illustrate states before the connection device 21c operates. FIGS. 10B, 10C, 10F, and 10G illustrate states after the connection device 21c operates. FIGS. 10D and 10H illustrate states after the connection device 21c returns. The states illustrated in FIGS. 10A to 10H correspond to the states illustrated in FIGS. 8A to 8H, which occur in the same timing.

In the present embodiment, the connection device 21c uses an electromagnet 21cn. The electromagnet 21cn is fixed to the drive wire W13. As is the case for the second embodiment, the connection device 21c illustrated in FIGS. 10A to 10H does not serve to connect the catheter unit 100 to the base unit 200.

In the present embodiment, the connection device 21c includes a sensor (not illustrated) serving as a tension detection unit and configured to detect the tension of the drive wire W13. The sensor detects the tension of the drive wire W13. When the tension stays in a normal range, the control unit 3 energizes the electromagnet 21cn. If the sensor detects an excessive force (exceeding a predetermined tension), the control unit 3 stops energizing the electromagnet 21cn. The connection device 21c assumes the state illustrated in FIGS. 10B and 10C from the state illustrated in FIG. 10A by cutting the electricity to the electromagnet 21cn. The other structures and operations of the connection device 21c are the same as those for the first and second embodiments, and the descriptions thereof are omitted.

The catheter 11 as configured above is inserted into or extracted from the object without causing damage to the object. If an excessive force occurs again, the user repeats the above procedure to avoid damage to the object and inserts the catheter 11 into the object or extracts the catheter 11 from the object.

Also in the present embodiment, when an excessive force applies to any of the first to ninth drive wires W1l to W33, the user takes the above-described actions to avoid damage to the object and continues to operate the catheter 11.

### Fourth Embodiment

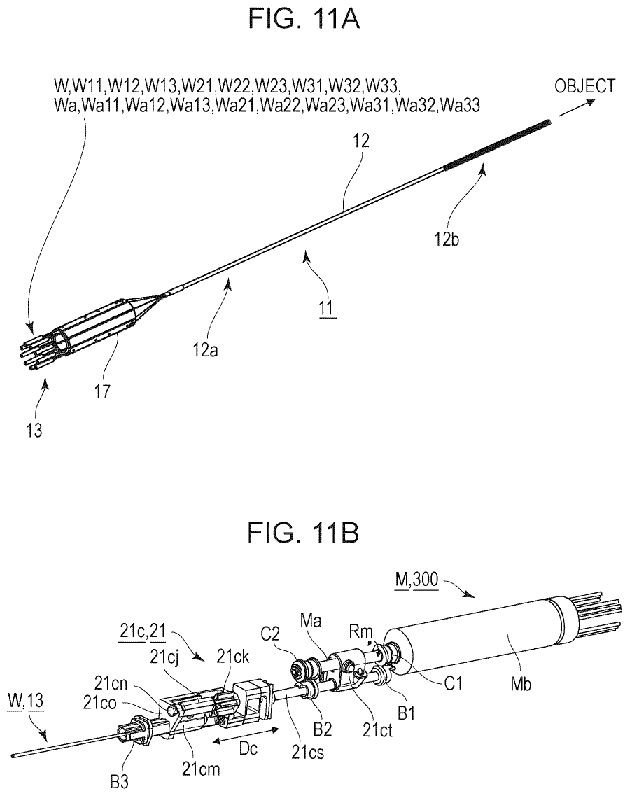

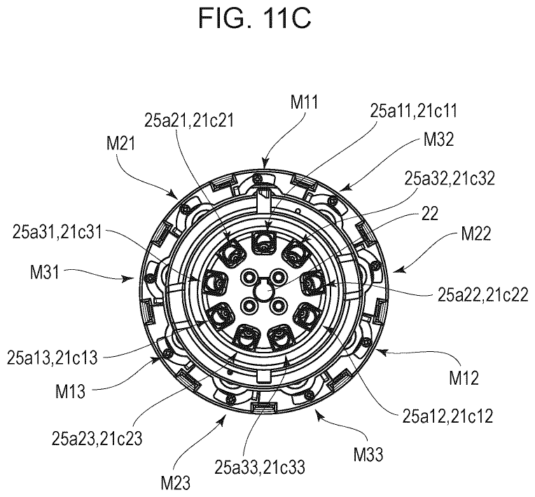

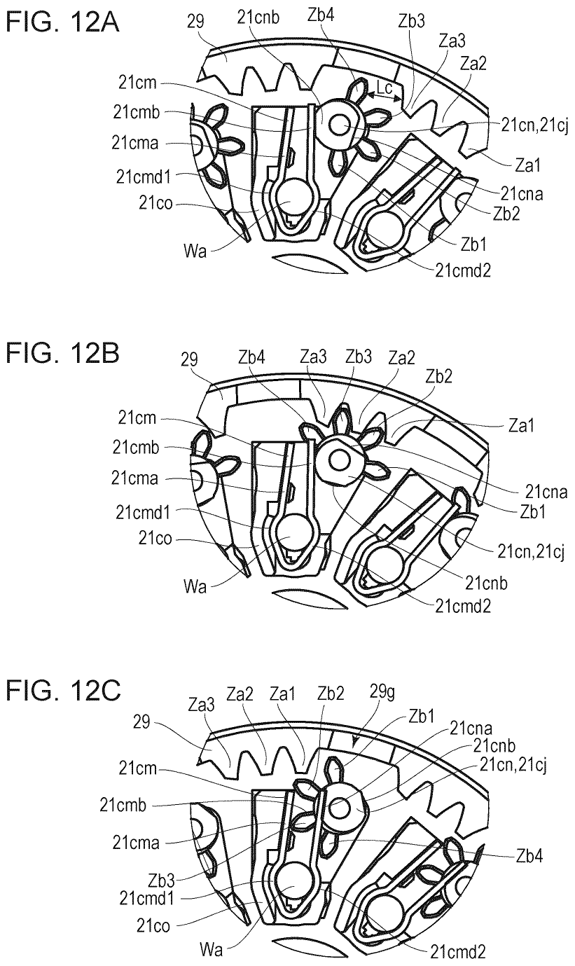

Referring to FIGS. 11A to 11C and FIGS. 12A to 12C, the following describes operation of a connection device 21c of the present embodiment when a drive wire W is connected to, and released from, the connection device 21c. FIG. 11A is a general view of the catheter 11.

FIG. 11B is a perspective view illustrating the drive source M, the connection device 21c, and the drive wire W13. FIG. 11C illustrates the base unit 200 as viewed in the attachment and detachment direction DE. FIG. 12A illustrates a state in which the drive wire W is connected. FIG. 12B illustrates a state in which the drive wire W is in the process of being released. FIG. 12C illustrates a state in which the drive wire W is released.

The drive wire W includes a retention member (or retention shaft or rod) Wa.

The connection device 21c includes a flat spring 21cm, which serves as a holding member for holding the retention member Wa of the drive wire W. The drive wire W passes through the insertion hole 25a and engages the connection device 21c. More specifically, the retention member Wa engages the flat spring 21cm. The flat spring 21cm can assume two states, in other words, a fixing state in which the flat spring 21cm pinches the retention member Wa and a releasing state in which the flat spring 21cm releases the retention member Wa.

The connection device 21c includes a pressing member 21cj. The pressing member 21cj includes a gear 21ck to mesh with an internal gear 29 and a cam 21cn serving as a pressing portion to press the flat spring 21cm. The cam 21cn is movable (rotatable) relative to the flat spring 21cm. The movement of the cam 21cn switches the state of the flat spring 21cm between the fixing state and releasing state.

The flat spring 21cm of the connection device 21c includes a fixed portion 21cma and a pressed portion 21cmb. As illustrated in FIG. 12A, which illustrates the fixing state, the fixed portion 21cma is fixed to a connection base 21co, and the pressed portion 21cmb is in contact with the cam 21cn of the pressing member 21cj. The flat spring 21cm also includes a first portion 21cmd1 and a second portion 21cmd2. When the catheter unit 100 is attached to the base unit 200, the retention member Wa is inserted between the first portion 21cmd1 and the second portion 21cmd2.

In the fixing state illustrated in FIG. 12A, the pressed portion 21cmb of the flat spring 21cm is pressed by a pressing surface 21cnb of the pressing member 21cj and is held in this state.

When the operation portion 400 is rotated in a releasing direction from the fixing state illustrated in FIG. 12A, a tooth Zb4 of the gear 21ck comes into contact with a tooth Za3 of the internal gear 29. When the operation portion 400 is further rotated in the releasing direction, the internal gear 29 rotates the cam 21cn as illustrated in FIG. 12B. Further rotation of the cam 21cn, as illustrated in FIG. 12C, brings a holding surface 21cna of the cam 21cn into contact with the pressed portion 21cmb of the flat spring 21cm, thereby releasing the drive wire W. The excessive force is removed by releasing the drive wire W. After the excessive force is removed, the user rotates the operation portion 400 in a fixation direction to fix the drive wire W again between the first portion 21cmd1 and the second portion 21cmd2.

The catheter 11 as configured above is inserted into or extracted from the object without causing damage to the object. If an excessive force occurs again, the user repeats the above procedure to avoid damage to the object and inserts the catheter 11 into the object or extracts the catheter 11 from the object.

Also in the present embodiment, when an excessive force applies to any of the first to ninth drive wires W1l to W33, the user takes the above-described actions to avoid damage to the object and continues to operate the catheter 11.

The connection device 21c of the present embodiment can serve to connect the catheter unit 100 to the base unit 200.

According to the one or more features of the present disclosure, the transmission of power can be resumed.

While one or more features of the present disclosure have been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

1. A continuum robot comprising:
a linear member having flexibility;
a driving unit configured to actuate the linear member;
a bendable portion configured to be bent by actuation of the linear member; and
a connection device configured to be connected to the linear member and to transmit actuation power of the driving unit to the linear member,
wherein the connection device and the linear member are configured to be reconnected after the connection device and the linear member that have been connected are disconnected from each other.

2. The continuum robot according to claim 1,
wherein the reconnection between the connection device and the linear member is made automatically.

3. The continuum robot according to claim 1, the continuum robot further comprising:
a detection unit configured to detect an amount of relative movement between the connection device and the linear member,
wherein after the connection device and the linear member that have been connected are disconnected from each other, the reconnection between the connection device and the linear member is made in accordance with the amount of relative movement detected by the detection unit.

4. The continuum robot according to claim 3,
wherein the detection unit is configured to detect the disconnection between the connection device and the linear member.

5. The continuum robot according to claim 1,
wherein after the connection device and the linear member are disconnected, the driving unit reconnects the connection device and the linear member by moving the connection device.

6. The continuum robot according to claim 1,
wherein the connection device and the linear member are connected by using a magnet.

7. The continuum robot according to claim 1,
wherein the connection device includes a first magnet, a connection member, and a second magnet, and
wherein the connection device and the linear member are connected in such a manner that the first magnet is in contact with the connection member, the second magnet is in contact with the connection member, and the second magnet is joined to the linear member.

8. The continuum robot according to claim 7,
wherein a contact force acting between the first magnet and the connection member is smaller than a contact force acting between the second magnet and the linear member.

9. The continuum robot according to claim 1, further comprising:
an elastic member urging the linear member in a direction of the linear member being connected to the connection device.

10. The continuum robot according to claim 1, further comprising:
a tension detection unit configured to detect tension of the linear member,
wherein the connection device and the linear member are connected to each other by using an electromagnet, and
wherein the electromagnet disconnects the connection device and the linear member from each other when the tension detection unit detects a predetermined amount of tension or more of the linear member.

11. The continuum robot according to claim 1,
wherein the disconnection and the reconnection between the connection device and the linear member are made manually.
